(19) United States
(12) Patent Application Publication
(10) Pub. No.: US 2026/0256396 A1
(43) Pub. Date: Sep. 3, 2026

(54) SYSTEMS AND METHODS FOR MONITORING DRIVING SESSIONS

(71) Applicant: RESMED DIGITAL HEALTH INC., San Diego, CA (US)

(72) Inventors: Jamie Graeme Wehbeh, Bella Vista, New South Wales (AU); Priyanshu Gupta, Sydney (AU); Genevieve Claire Madafiglio, Sydney (AU)

(73) Assignee: RESMED DIGITAL HEALTH INC., San Diego, CA (US)

(21) Appl. No.: 18/879,624

(22) PCT Filed: Jun. 29, 2023
(86) PCT No.: PCT/AU2023/050604

§ 371 Date: Dec. 27, 2024

Related U.S. Application Data
(60) Provisional application No. 63/357,550, filed on Jun. 30, 2022.

(51) Int. Cl. A61B 5/18; A61B 5/00; A61B 5/0205

(52) U.S. Cl. CPC A61B 5/18; A61B 5/0077; A61B 5/0205; A61B 5/4818; A61B 5/7275

(57) ABSTRACT

A method for determining a likelihood that an individual has or will develop a condition comprises generating, during a plurality of driving sessions, data associated with the plurality of driving sessions. The individual is located within a vehicle during at least a portion of each of the plurality of driving sessions. The method further comprises analyzing the data and determining, based at least in part on the analyzed data, a risk factor for the individual associated with the condition. The data associated with the plurality of driving sessions includes one or more parameters. Analyzing the data can include determining a value of each of the parameters, a change in the value of the parameters between two driving sessions, and/or an average rate of change in the value of the parameters across multiple driving sessions.

### CROSS-REFERENCE TO RELATED APPLICATIONS

This application claims the benefit of, and priority to, U.S. Provisional Patent Application No. 63/357,550 filed on Jun. 30, 2022, which is hereby incorporated by reference herein in its entirety.

### TECHNICAL FIELD

The present disclosure relates generally to systems and methods for monitoring driving sessions, and more particularly, to systems and methods predicting the likelihood that an individual has or will develop a condition based at least in part on data collected during one or more driving sessions.

### BACKGROUND

Many individuals suffer from sleep-related and/or respiratory-related disorders such as, for example, Sleep Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (e.g., difficulty initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These disorders are often treated using a respiratory therapy system (e.g., a continuous positive airway pressure (CPAP) system), which delivers pressurized air to aid in preventing the individual's airway from narrowing or collapsing during sleep. It can sometimes be difficult to determine if an individual has any of these conditions, or if the individual is at risk for developing any of these conditions. Thus, new systems and methods are needed for determining whether the individual has any of these conditions and/or is at risk of developing any of these conditions. The present disclosure is directed to solving these and other problems.

### SUMMARY

According to some implementations of the present disclosure, a method for determining a likelihood that an individual has or will develop a condition comprises generating, during a plurality of driving sessions, data associated with the plurality of driving sessions. The individual is located within a vehicle during at least a portion of each of the plurality of driving sessions. The method also includes analyzing the data. The method also includes determining, based at least in part on the analyzed data, a risk factor for the individual that is associated with the condition. The risk factor can include a percentage likelihood that the individual will develop the condition, an estimated time period within which the individual will develop the condition, a percentage likelihood that the individual currently has the condition, or any combination thereof. The data associated with the plurality of driving sessions includes one or more parameters. Analyzing the data can include determining a value of each of the parameters, a change in the value of the parameters between two driving sessions, and/or an average rate of change in the value of the parameters across multiple driving sessions.

According to some implementations of the present disclosure, a system for determining a likelihood that an individual has or will develop a condition comprises an electronic interface, a control system, and a memory. The electronic interface is configured to receive data associated with a plurality of driving sessions. The memory stores machine-readable instructions. The control system includes one or more processors configured to execute the machine-readable instructions to execute a method. The method includes analyzing the data and determining, based at least in part on the analyzed data, a risk factor for the individual that is associated with the condition. The risk factor can include a percentage likelihood that the individual will develop the condition, an estimated time period within which the individual will develop the condition, a percentage likelihood that the individual currently has the condition, or any combination thereof.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3 illustrates an exemplary timeline for a sleep session, according to some implementations of the present disclosure;

FIG. 4 illustrates an exemplary hypnogram associated with the sleep session of FIG. 3, according to some implementations of the present disclosure;

FIG. 5A is a perspective view of an individual operating a vehicle while fully awake, according to some implementations of the present disclosure;

FIG. 5B is a perspective view of an individual operating a vehicle while showing signs of daytime tiredness, according to some implementations of the present disclosure;

FIG. 5C is a perspective view of an individual operating a vehicle while showing severe signs of daytime tiredness, according to some implementations of the present disclosure; and

FIG. 6 is a flow diagram of a method for determining a likelihood that an individual has or will develop a condition, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

The present disclosure is described with reference to the attached figures, where like reference numerals are used throughout the figures to designate similar or equivalent elements. The figures are not drawn to scale, and are provided merely to illustrate the instant disclosure. Several aspects of the disclosure are described below with reference to example applications for illustration.

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). CSA results when the brain temporarily stops sending signals to the muscles that control breathing. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfil the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

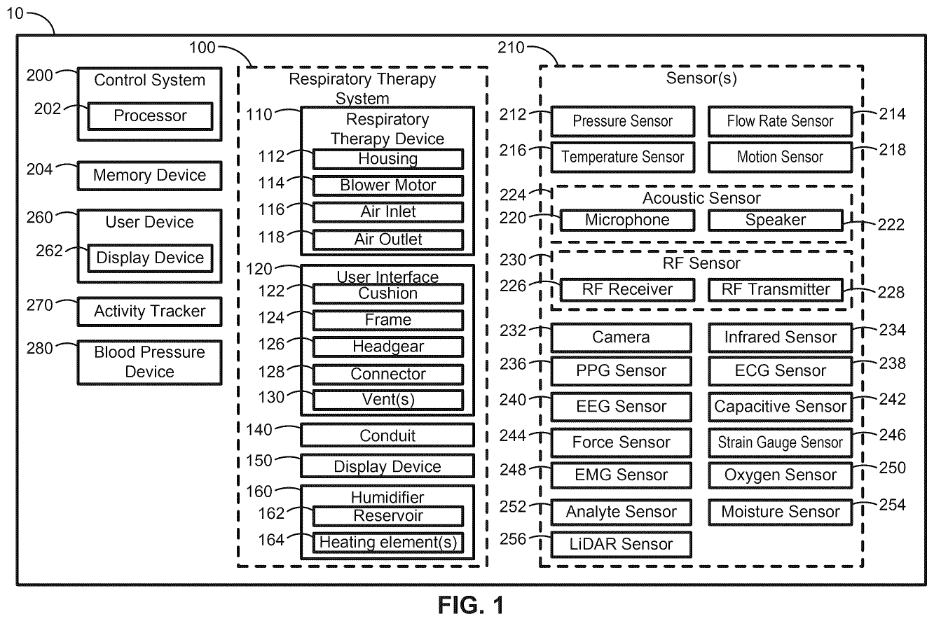

Referring to FIG. 1, a system 10, according to some implementations of the present disclosure, is illustrated. The system 10 can include a respiratory therapy system 100, a control system 200, a memory device 204, and one or more sensors 210. The system 10 may additionally or alternatively include a user device 260, an activity tracker 270, and a blood pressure device 280. The system 10 can be used to analyze data from a plurality of driving sessions of an individual, in order to determine the likelihood that the individual has developed a condition (such as OSB or SDB) or will develop a condition (such as OSA or SDB).

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

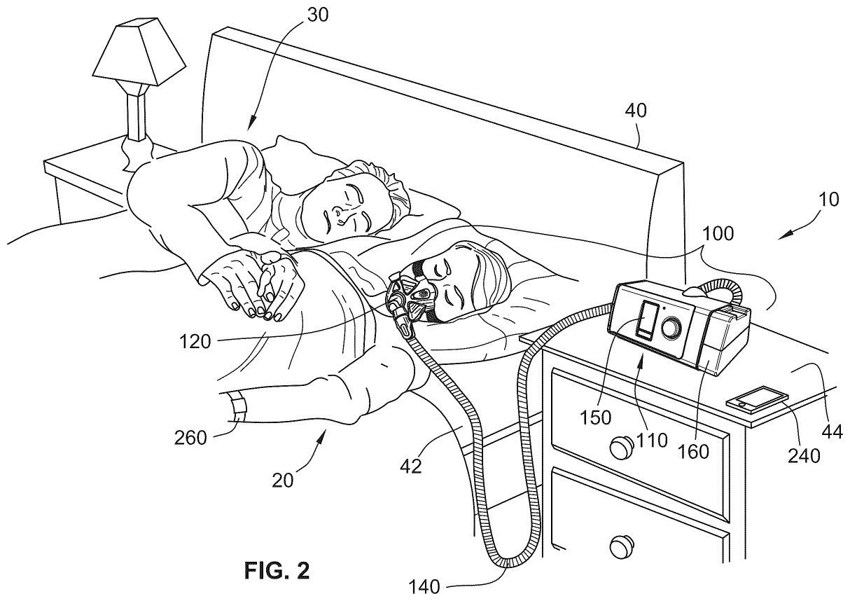

As shown in FIG. 2, the respiratory therapy system 100 can be used to treat a user 20. In this example, the user 20 of the respiratory therapy system 100 and a bed partner 30 are located in a bed 40 and are laying on a mattress 42. The user interface 120 can be worn by the user 20 during a sleep session. The respiratory therapy system 100 generally aids in increasing the air pressure in the throat of the user 20 to aid in preventing the airway from closing and/or narrowing during sleep. The respiratory therapy device 110 can be positioned on a nightstand 44 that is directly adjacent to the bed 40 as shown in FIG. 2, or more generally, on any surface or structure that is generally adjacent to the bed 40 and/or the user 20.

Referring back to FIG. 1, the respiratory therapy device 110 is generally used to generate pressurized air that is delivered to a user (e.g., using one or more motors that drive one or more compressors). In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6cmH₂O, at least about 10 cmH₂O, at least about 20 cmH₂O, between about 6cmH₂ O and about 10 cmH₂O, between about 7 cmH₂O and about 12 cmH₂O, etc. The respiratory therapy device 110 can also deliver pressurized air at a predetermined flow rate between, for example, about −20 L/min and about 150 L/min, while maintaining a positive pressure (relative to the ambient pressure).

The respiratory therapy device 110 includes a housing 112, a blower motor 114, an air inlet 116, and an air outlet 118. The blower motor 114 is at least partially disposed or integrated within the housing 112. The blower motor 114 draws air from outside the housing 112 (e.g., atmosphere) via the air inlet 116 and causes pressurized air to flow through the humidifier 160, and through the air outlet 118. In some implementations, the air inlet 116 and/or the air outlet 118 include a cover that is moveable between a closed position and an open position (e.g., to prevent or inhibit air from flowing through the air inlet 116 or the air outlet 118). The housing 112 can also include a vent to allow air to pass through the housing 112 to the air inlet 116. As described below, the conduit 140 is coupled to the air outlet 118 of the respiratory therapy device 110.

The user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

The user interface 120 can include, for example, a cushion 122, a frame 124, a headgear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners). The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The vent 130 can be used for permitting the escape of carbon dioxide and other gases exhaled by the user 20. The user interface 120 generally can include any suitable number of vents (e.g., one, two, five, ten, etc.).

As shown in FIG. 2, in some implementations, the user interface 120 is a facial mask (e.g., a full face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

Referring back to FIG. 1, the conduit 140 (also referred to as an air circuit or tube) allows the flow of air between components of the respiratory therapy system 100, such as between the respiratory therapy device 110 and the user interface 120. In some implementations, there can be separate limbs of the conduit for inhalation and exhalation. In other implementations, a single limb conduit is used for both inhalation and exhalation.

The conduit 140 includes a first end that is coupled to the air outlet 118 of the respiratory therapy device 110. The first end can be coupled to the air outlet 118 of the respiratory therapy device 110 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end can include an electrical contact that is electrically coupled to the respiratory therapy device 110 to power the one or more heating elements of the conduit 140. For example, the electrical contact can be electrically coupled to an electrical contact of the air outlet 118 of the respiratory therapy device 110. In this example, electrical contact of the conduit 140 can be a male connector and the electrical contact of the air outlet 118 can be female connector, or, alternatively, the opposite configuration can be used.

The display device 150 is generally used to display image(s) including still images, video images, or both and/or information regarding the respiratory therapy device 110. For example, the display device 150 can provide information regarding the status of the respiratory therapy device 110 (e.g., whether the respiratory therapy device 110 is on/off, the pressure of the air being delivered by the respiratory therapy device 110, the temperature of the air being delivered by the respiratory therapy device 110, etc.) and/or other information (e.g., a sleep score and/or a therapy score, also referred to as a myAir™ score, such as described in WO 2016/061629 and U.S. Patent Pub. No. 2017/0311879, which are hereby incorporated by reference herein in their entireties, the current date/time, personal information for the user 20, etc.). In some implementations, the display device 150 acts as a human-machine interface (HMI) that includes a graphic user interface (GUI) configured to display the image(s) as an input interface. The display device 150 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 110.

The humidifier 160 is coupled to or integrated in the respiratory therapy device 110 and includes a reservoir 162 for storing water that can be used to humidify the pressurized air delivered from the respiratory therapy device 110. The humidifier 160 includes a one or more heating elements 164 to heat the water in the reservoir to generate water vapor. The humidifier 160 can be fluidly coupled to a water vapor inlet of the air pathway between the blower motor 114 and the air outlet 118, or can be formed in-line with the air pathway between the blower motor 114 and the air outlet 118. For example, air flows from the air inlet 116 through the blower motor 114, and then through the humidifier 160 before exiting the respiratory therapy device 110 via the air outlet 118.

While the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other.

The memory device 204 stores machine-readable instructions that are executable by the processor 202 of the control system 200. The memory device 204 can be any suitable computer readable storage device or media, such as, for example, a random or serial access memory device, a hard drive, a solid state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to 110 and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct).

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 202 and/or memory device 204 can receive data (e.g., physiological data and/or audio data) from the one or more sensors 210 such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260.

The one or more sensors 210 include a pressure sensor 212, a flow rate sensor 214, temperature sensor 216, a motion sensor 218, a microphone 220, a speaker 222, a radio-frequency (RF) receiver 226, a RF transmitter 228, a camera 232, an infrared (IR) sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a Light Detection and Ranging (LiDAR) sensor 256, or any combination thereof. Generally, each of the one or more sensors 210 are configured to output sensor data that is received and stored in the memory device 204 or one or more other memory devices.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the IR sensor 234, the PPG sensor 236, the ECG sensor 238, the EEG sensor 240, the capacitive sensor 242, the force sensor 244, the strain gauge sensor 246, the EMG sensor 248, the oxygen sensor 250, the analyte sensor 252, the moisture sensor 254, and the LiDAR sensor 256, more generally, the one or more sensors 210 can include any combination and any number of each of the sensors described and/or shown herein.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

The one or more sensors 210 can be used to generate, for example, physiological data, audio data, or both. Physiological data generated by one or more of the sensors 210 can be used by the control system 200 to determine a sleep-wake signal associated with the user 20 during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleet stage, an apnea-hypopnea index (AHI), pressure settings of the respiratory therapy device 110, or any combination thereof. The one or more events can include snoring, apneas, central apneas, obstructive apneas, mixed apneas, hypopneas, a mask leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 210, or from other types of data.

The pressure sensor 212 outputs pressure data that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof.

The flow rate sensor 214 outputs flow rate data that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20, a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 alternatively or additionally generates one or more signals representing bodily movement of the user, from which may be obtained a signal representing a sleep state of the user; for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones

The speaker 222 outputs sound waves that are audible to a user of the system 10 (e.g., the user 20 of FIG. 2). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260.

The microphone 220 and the speaker 222 can be used as separate devices. In some implementations, the microphone 220 and the speaker 222 can be combined into an acoustic sensor 224 (e.g., a SONAR sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human ear (e.g., below 20 Hz or above around 18 kHz) so as not to disturb the sleep of the user 20 or the bed partner 30. Based at least in part on the data from the microphone 220 and/or the speaker 222, the control system 200 can determine a location of the user 20 and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 228 generates and/or emits radio waves having a predetermined frequency and/or a predetermined amplitude (e.g., within a high frequency band, within a low frequency band, long wave signals, short wave signals, etc.). The RF receiver 226 detects the reflections of the radio waves emitted from the RF transmitter 228, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g. a RADAR sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user, to determine air flow of the mouth and/or nose of the user, to determine a time when the user enters the bed, and to determine a time when the user exits the bed. In some implementations, the camera 232 includes a wide angle lens or a fish eye lens.

The IR sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated in the user interface 120 and/or the associated headgear (e.g., straps, etc.).

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 252 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 252 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The LiDAR sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 10, including the respiratory therapy device 110, the user interface 120, the conduit 140, the humidifier 160, the control system 200, the user device 260, the activity tracker 270, or any combination thereof. For example, the microphone 220 and the speaker 222 can be integrated in and/or coupled to the user device 260 and the pressure sensor 212 and/or flow rate sensor 214 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 210, or from other types of data.

The user device 260 includes a display device 262. The user device 260 can be, for example, a mobile device such as a smart phone, a tablet, a gaming console, a smart watch, a laptop, or the like. Alternatively, the user device 260 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10.

In some implementations, the system 10 also includes the activity tracker 270. The activity tracker 270 is generally used to aid in generating physiological data associated with the user. The activity tracker 270 can include one or more of the sensors 210 described herein, such as, for example, the motion sensor 218 (e.g., one or more accelerometers and/or gyroscopes), the PPG sensor 236, and/or the ECG sensor 238. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIG. 2, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 10 also includes the blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIG. 2, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 maybe positioned external to the respiratory therapy system 100, coupled directly or indirectly to the user interface 120, coupled directly or indirectly to a headgear associated with the user interface 120, or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp chirp, triangle, sinewave), other schemes such as PSK, FSK etc., pulsed continuous wave, and/or spread in ultra wideband ranges (which may include spreading, PRN codes or impulse systems).

While the control system 200 and the memory device 204 are described and shown in FIG. 1 as being a separate and distinct component of the system 10, in some implementations, the control system 200 and/or the memory device 204 are integrated in the user device 260 and/or the respiratory therapy device 110. Alternatively, in some implementations, the control system 200 or a portion thereof (e.g., the processor 202) can be located in a cloud (e.g., integrated in a server, integrated in an Internet of Things (IOT) device, connected to the cloud, be subject to edge cloud processing, etc.), located in one or more servers (e.g., remote servers, local servers, etc., or any combination thereof.

While system 10 is shown as including all of the components described above, more or fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

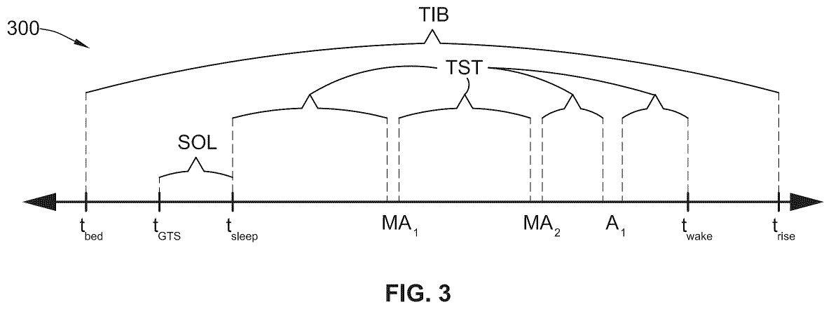

Referring now to FIG. 3, as used herein, a sleep session can be defined multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user has laid or sat down in the bed (or another area or object on which they intend to sleep), and has turned on the respiratory therapy device 110 and donned the user interface 120. The sleep session can thus include time periods (i) when the user is using the respiratory therapy system 100, but before the user attempts to fall asleep (for example when the user lays in the bed reading a book); (ii) when the user begins trying to fall asleep but is still awake; (iii) when the user is in a light sleep (also referred to as stage 1 and stage 2 of non-rapid eye movement (NREM) sleep); (iv) when the user is in a deep sleep (also referred to as slow-wave sleep, SWS, or stage 3 of NREM sleep); (v) when the user is in rapid eye movement (REM) sleep; (vi) when the user is periodically awake between light sleep, deep sleep, or REM sleep; or (vii) when the user wakes up and does not fall back asleep.

The sleep session is generally defined as ending once the user removes the user interface 120, turns off the respiratory therapy device 110, and gets out of bed. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user, ending when the respiratory therapy device 110 stops supplying the pressurized air to the airway of the user, and including some or all of the time points in between, when the user is asleep or awake.

FIG. 3 illustrates an exemplary timeline 300 for a sleep session. The timeline 300 includes an enter bed time (t_{bed}), a go-to-sleep time (t_{GTS}), an initial sleep time (tₛₗₑₑₚ), a first micro-awakening MA₁, a second micro-awakening MA₂, an awakening A, a wake-up time (t_{wake}), and a rising time (tᵣᵢₛₑ).

The enter bed time t_{bed} is associated with the time that the user initially enters the bed (e.g., bed 40 in FIG. 2) prior to falling asleep (e.g., when the user lies down or sits in the bed). The enter bed time t_{bed} can be identified based at least in part on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 260, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based at least in part on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based at least in part on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based at least in part on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based at least in part on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based at least in part on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, as shown in the timeline 300, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

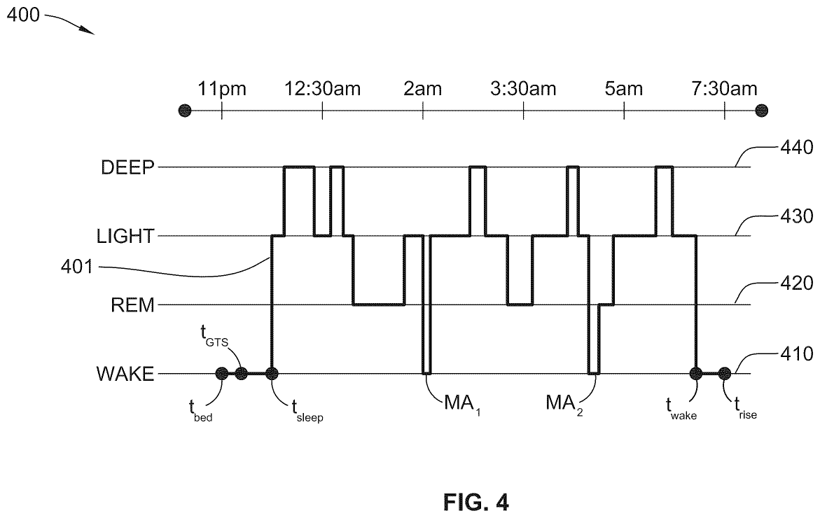

Referring to FIG. 4, an exemplary hypnogram 400 corresponding to the timeline 300 of FIG. 3, according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based at least in part on physiological data associated with the user (e.g., generated by one or more of the sensors 210 described herein). The sleep-wake signal can be indicative of one or more sleep stages, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration amplitude ratio, an inspiration-expiration duration ratio, a number of events per hour, a pattern of events, or any combination thereof. Information describing the sleep-wake signal can be stored in the memory device 204.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.) The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based at least in part on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 210 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based at least in part on, for example, data generated by the motion sensor 218, the microphone 220, the camera 232, or any combination thereof. The go-to-sleep time can be determined based at least in part on, for example, data from the motion sensor 218 (e.g., data indicative of no movement by the user), data from the camera 232 (e.g., data indicative of no movement by the user and/or that the user has turned off the lights), data from the microphone 220 (e.g., data indicative of the using turning off a TV), data from the user device 260 (e.g., data indicative of the user no longer using the user device 260), data from the pressure sensor 212 and/or the flow rate sensor 214 (e.g., data indicative of the user turning on the respiratory therapy device 110, data indicative of the user donning the user interface 120, etc.), or any combination thereof.

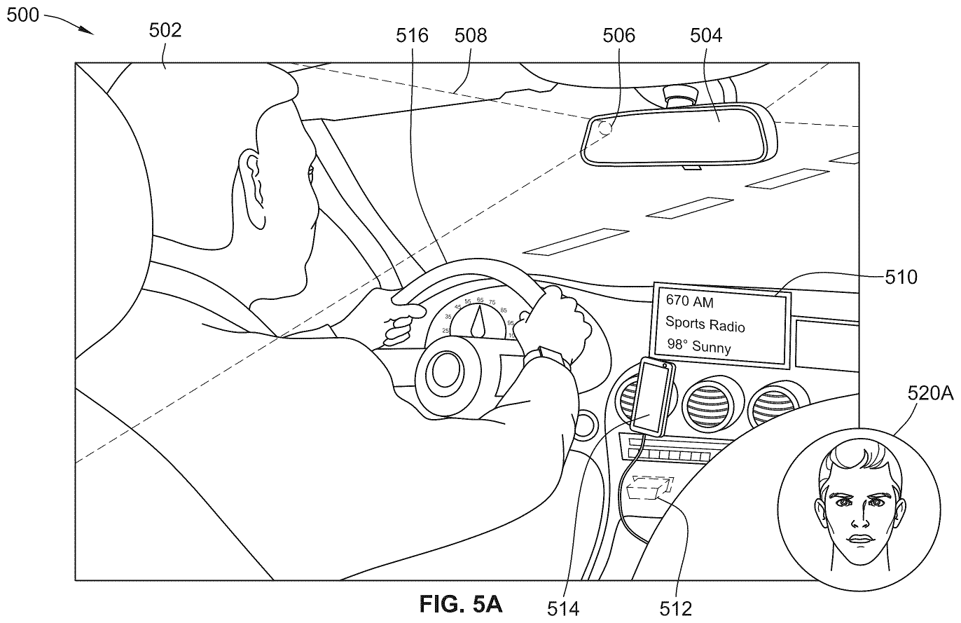

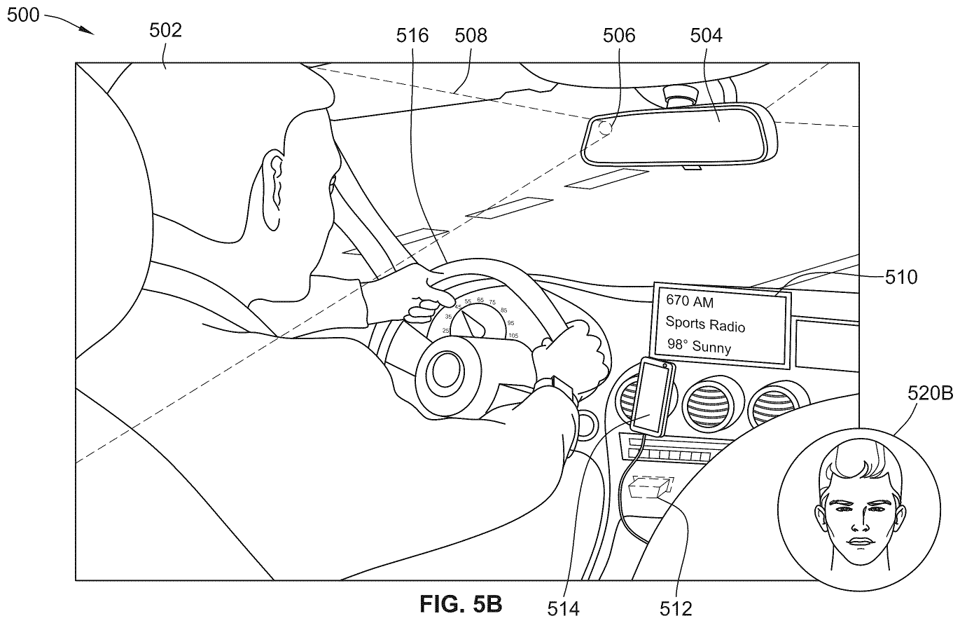

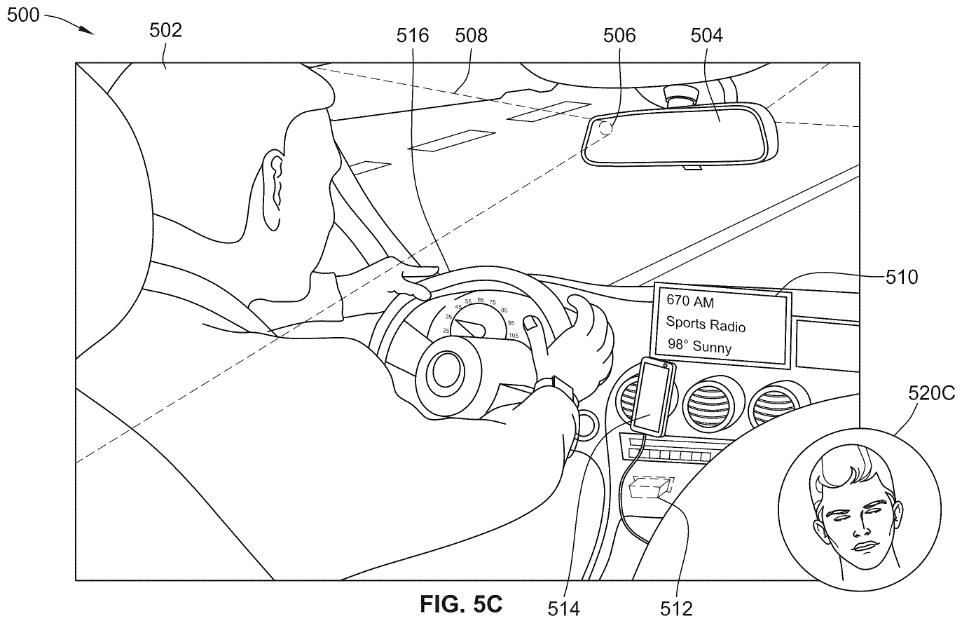

Referring now to FIGS. 5A, 5B, and 5C, data that is related to the use of a vehicle by an individual (such as the user 20) can be analyzed to determine the individual's risk factor for developing and/or having a condition. In some cases, the condition is sleep-disordered breathing (SDB), such as obstructive sleep apnea (OSA). In other cases, the condition includes allergies, a cold, a dry throat, a sore throat, a viral infection, pneumonia, chronic obstructive pulmonary disease (COPD), asthma, stroke, enlarged tonsils, high blood pressure, low blood pressure, or any combination thereof. The present disclosure will generally refer to either “the condition” or specifically refer to SDB. However, those of skill in the art will understand that references to the condition, or specifically to SDB, can generally refer to any number of conditions that the individual can develop, not just SDB.

Generally, determining whether an individual already has these conditions (or other conditions) or is at risk for developing these conditions (or other conditions) will be referred to as determining the individual's risk factor for these condition (or other conditions). FIGS. 5A, 5B, and 5C illustrate how data related to the individual's use of the vehicle can be collected in order to determine the individual's risk factor for these conditions.

FIG. 5A-5C shows an individual 502 operating a vehicle 500, which can be considered to be part of the system 10 of FIG. 1 in some implementations. The vehicle 500 includes a rear-view mirror 504 with an embedded image sensor 506. The image sensor 506 can be a camera (such as the camera 232 of the system 10) an IR sensor (such as the IR sensor 234 of the system 10), or various other types of image sensors. The image sensor 506 is operable to generate image data that is reproducible as one or more images and/or one or more videos of the individual 502 within the vehicle 500. The image sensor 506 has a field of view 508 that will generally include the individual 502. Objects within the field of view 508 will generally be shown in any images and/or videos generated from the image data. The rear-view mirror 504 may include an additional image sensor (not shown) that faces outward away from the individual 502, towards the road (or whatever surface the vehicle 500 is traveling on). Image data from this additional image sensor can be used to analyze movement of the vehicle.

The vehicle 500 further includes a center console 510, a data port 512, and a mobile device 514 mounted in view of the individual 502, and a data port 512. In the illustrated implementation, the center console 510 is a display device that displays various information and can be used by the individual 502 to adjust various settings in the vehicle 500, such as the climate control settings and audio settings. The center console 510 can have other forms as well.

In the illustrated implementation, the mobile device 514 (which could be the same as or similar to the user device 260 of the system 10) is the individual 502's smartphone, and can be mounted on the dashboard of the vehicle 500 so that the individual 502 can use the mobile device 514 for various purposes, for example as a GPS device. The mobile device 514 may include additional sensors (e.g., an image sensor, an optical sensor, a vibration sensor, etc.) that can generate data associated with the individual 502's operation of the vehicle.

The data port 512 allows access to data stored in an onboard control system of the vehicle 500. As the vehicle operates, a variety of different data can be stored in the vehicle 500's control system. This data can include data related to the operation of the vehicle 500 (e.g., speed, acceleration, tire movement, movement of the vehicle 500, location of the vehicle 500, etc.), data related to the health of the vehicle 500, data related to various settings and/or features of the vehicle 500 (e.g., climate control settings, position of the driver's seat, activation of heated or cooled seats, activation of a massage feature in the seats, etc.). A device can be electronically coupled to the data port 512 (e.g., a wired connection, a wireless connection, or both) to collect data that is stored in the vehicle 500's control system.

Any suitable device can be electronically coupled to the data port 512 to collect the data, such as the mobile device 514, the user device 260 of system 10 (if the user device 260 is different from the mobile device 514), the control system 200 of system 10, and other devices. The vehicle 500 may include additional or alternative sensors that can be used to analyze the individual 502 and the individual 502's use of the vehicle 500. In some cases, some or all of these sensors store data in the vehicle 500's control system where it can be gathered via the data port 512. In other cases, some or all of these sensors do not store data in the control system of the vehicle, but instead store the data locally and/or transmit the data to other devices (such as the mobile device 514, the user device 260 of system 10, the control system 200 of system 10), etc.) separate from the vehicle 500's control system.

Data related to the operation of the vehicle 500 by the individual 502 can be analyzed to aid in determining the risk factor of the individual 502. For example, individuals with SDB (or at risk of developing SDB) may exhibit signs if being tired during the day when they are driving. Image data generated by the image sensor 506 (or any other image sensor) can be analyzed to determine if the individual 502 is exhibiting signs of being tired or sleepy, such as eyes blinking more rapidly than usual, head nodding, etc. The image data can also be analyzed to determine characteristics related to the individual, such as the individual's posture, weight, neck circumference, waist circumference, etc. The operation of the vehicle 500 can also be analyzed to aid in determining the risk factor of the individual 502. For example, if the individual 502 is tired during the daytime, the individual may play their radio louder, activate the massage function of their seat, activate the heating or cooling setting of their seat, and other actions. Data showing these actions can be generated and analyzed to aid in determining the risk factor of the individual 502. In another example, the individual 502 that is tired during the day due to SBD may drive erratically, such as by swerving in and out of their line, braking more aggressively than an average driver, etc. Data related to the operation of the vehicle can be analyzed to aid in determining the risk factor of the individual 502.

FIG. 5A shows an inset view 520A of the individual 502 when the individual 502 is fully alert. Generally, the inset view 520A is an example of an image and/or video generated from the image data of the image sensor 506. As can be seen in the inset view 520A, the individual 502's head is up and eyes are open, indicating that the individual 502 is not exhibiting any signs of tiredness. The individual 502 also has their hands at standard positions on a steering wheel 516 of the vehicle 500, and is keeping the vehicle 500 within the lane on the road. Thus, analysis of the data resulting from the scenario in FIG. 5A may indicate that the risk factor of the individual 502 is low.

In FIG. 5B, the individual 502 is showing signs of tiredness. As can be seen in the inset view 520B, the individual 502's head has begun to droop, and the individual 502's eyes are semi-closed. The individual 502 has begun to inadvertently rotate the steering wheel 516 clockwise, such that the vehicle 500 has begun to draft out of the line. The individual 502 has also begun to inadvertently reduce the speed of the vehicle 500. The image data generated by the image sensor 506 (or any other image sensor) can be analyzed to detect the movement of the individual 502's head and eyes, which can be used to determine the individual 502's risk factor. Additional data related to the movement of the vehicle can be obtained via the data port 512, which can also be used to determine the individual 502's risk factor.

In FIG. 5C, the individual 502 is showing more sever signs of tiredness. As shown in the inset view 520C, the individual 502's head is tilted toward the side, and the individual 502's eyes are closed. The individual has also continued to inadvertently rotate the steering wheel 514 further clockwise, such that the vehicle 500 has continued to veer out of the lane. The individual 502 has continued to inadvertently reduce the speed of the vehicle 500 as well. Similar to FIG. 5B, image data generated by the image sensor 506 and data related to the movement of the vehicle can be analyzed to determine the individual 502's risk factor for a conditions such as SDB.

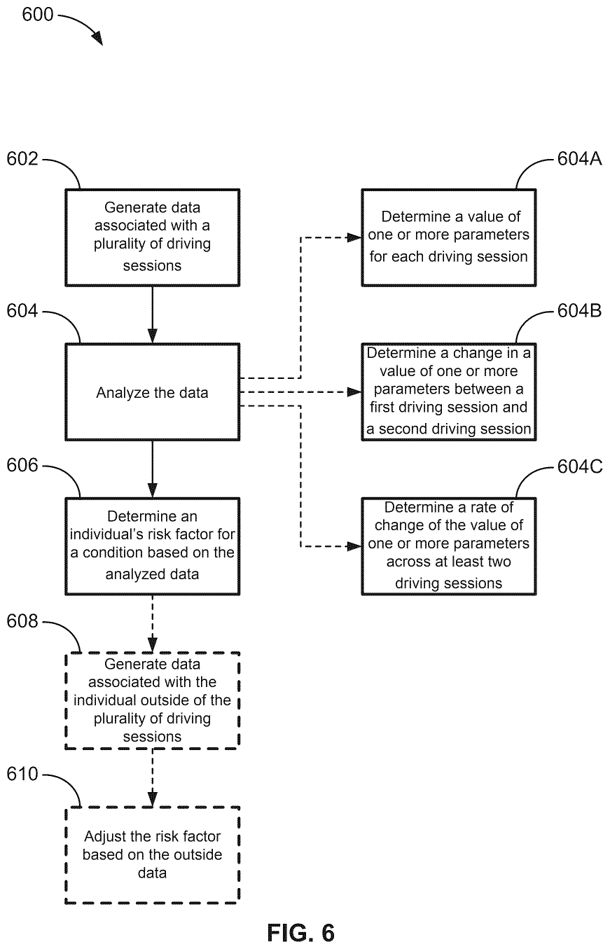

FIG. 6 illustrates a method 600 for determining the likelihood that an individual has a condition based at least in part on data associated with operation of a vehicle (which could be the same as or similar to vehicle 500. The condition can be a sleep apnea-related condition, such as SDB or OSA. The condition could also be any other kind of condition, including allergies, a cold, a dry throat, a sore throat, a viral infection, pneumonia, chronic obstructive pulmonary disease (COPD), asthma, stroke, enlarged tonsils, high blood pressure, low blood pressure, or any combination thereof

Method 600 may utilize data generated by a variety of different sensors, such as any of the sensors 210 of system 10. Method 600 may also utilize data generated by a respiratory therapy device (such as respiratory therapy device 110) of a respiratory therapy system (such as respiratory therapy system 100), data generated by a user device (such as user device 260 of system 10), data generated by an activity tracker (such as activity tracker 270 of system 10), data generated by a blood pressure device (such as blood pressure device 280 of system 10), and/or data from any other source. Method 600 can be implemented using a control system, such as the control system 200 of system 10. Portions of method 600 may be implemented using additional or alternative devices as sell, such as the individual's mobile device, laptop computer, tablet computer, etc., all of which could be the user device 260 of system 10.

At step 602 of method 600, data associated with a plurality of driving sessions is generated. During a driving session, the individual whose risk factor is being determined is generally located within the vehicle. In some or all of the driving sessions, the individual sits in the driver's seat and is operating the vehicle. However, the individual does not need to be the person operating the vehicle. In some or all of the driving sessions, the individual can be a passenger in the vehicle, while another person operates the vehicle (or while the vehicle operates itself, if the vehicle is a self-driving vehicle).

A driving session can be defined in a number of different ways. In some cases, a driving sessions begins when the individual enters the vehicle and/or when the vehicle leaves its origin (such as the individual's house), and ends when the individual vehicle arrives at its destination (such as the individual's workplace) and/or when the individual exits the vehicle. In some implementations, the driving session can include only the time during this trip when the vehicle is actually being operated. Thus, if the vehicle stops anywhere and is shut off (such as at a gas station or rest station), the period of time spent at this location is not considered to be part of the driving session, and data associated with this period of time will not be analyzed.

However, in other implementations, the driving session includes all of the time between the vehicle leaving its origin and arriving at its destination, regardless of how many stops the vehicle makes during the trip. In still other implementations, time periods where the vehicle stops are excluded from the driving session only if the vehicle is stopped for a threshold amount of time. Thus, if the individual spends 5 minutes at a gas station to replenish the gas tank of the vehicle and then continues on toward the destination, this time period may be considered to be part of the driving session. However, if the individual makes an extended stop at some location (e.g., 20 minutes, 1 hour, etc.), this time period is excluded. In some cases, the trip between the origin and the stop is a first driving session, and the trip between the stop and the destination is a second driving session. In other cases, the trip between the origin and the destination is a single driving session, and data associated with the period of time when the vehicle was at the stop is excluded.

Generally, stops made in service of traffic laws when the vehicle is still being operated (e.g., the engine is on, but the vehicle is either in park, or is not moving due to the driver depressing the brake pedal) are not excluded from the driving session. Thus, if the vehicle is stopped at a stoplight for a minute, data associated with that minute of time is still considered to be part of the driving session, even though the vehicle was not moving. However, in some implementations, this data could be excluded from the driving session. Thus, the driving session could include only time when the vehicle is actually moving, or could include time when the vehicle is moving and time when the vehicle is stopped.

The data can be generated in any suitable fashion. In some cases, as discussed herein with respect to FIGS. 5A-5C, the data can be generated by sensors or other components of the vehicle itself, and then downloaded from the vehicle's control system. In some cases, additional or alternative data can be generated using a device of the individual, such as a smartphone, smartwatch, etc.

Step 604 of method 600 includes analyzing the data that was generated during the plurality of driving sessions. In some implementations, the data associated with the plurality or driving sessions includes one or more parameters that are associated with each of the plurality of driving sessions. These parameters can be analyzed to determine the individual's risk factor. The parameters can generally be grouped into four different categories as detailed below. However, other parameters not mentioned herein, and other parameters that do not necessarily fit into any of the four below categories can also be analyzed.

The first category of parameters includes parameters that are associated with the body of the individual. These parameters can include the posture of the individual, the weight of the individual, the neck circumference of the individual, the waist circumference of the individual, a ratio of the individual's neck circumference to the individual's waist circumference, an amount of eye movement of the individual (e.g., whether the individual is focused on the road), a speed of the eye movement of the individual (e.g., how rapidly the individual looks around), amount of blinking of the individual, an amount of any head movement of the individual, a speed of any head movement of the individual, a direction of any head movement of the individual, an amount of body movement of the individual, a weight distribution of the individual in a seat of the vehicle (e.g., whether the individual's weight is forward as the individual tries to stay awake, whether the individual's weight is reclined which could indicate tiredness, etc.), the strength and/or position of the individual's grip on the vehicle's steering wheel, a movement frequency of the individual's grip on the vehicle's steering wheel, and other parameters.

Generally, these parameters can all be an indication that the individual is suffering from SDB, or is at risk of developing SDB. A large weight, neck circumference, waist circumference, and neck circumference to waist circumference ratio can all be indications that the individual is suffering from SDB or at risk of developing SDB. Certain movements of the individual's head (e.g., nodding up and down while driving), eyes (e.g., blinking more frequently to stay awake), and/or body (fidgeting and/or restless movements such as continual or near-continual movement of the individual's arms and legs) can indicate that the individual is tired during the day, which can be an indication that the individual is suffering from SDB or at risk from developing SDB. A tired individual may also move their eyes faster or slower, and may tend to focus their eyesight on a target for longer or shorter periods. The individual's grip on the steering wheel can also indicate that the individual is tired during the day. For example, a weak grip strength, a certain grip position on the steering wheel (e.g., hands at 10:00 and 2:00, one hand on the bottom, hands and/or wrists at certain angles, etc.), and a frequently changing grip position can all indicate that the individual is tired and is attempting to stay awake. Generally, any combination of the above parameters and/or other parameters can be determined to aid in determining the individual's risk factor.

The second category of parameters includes parameters that are associated with the environment around the individual during the plurality of driving sessions. These parameters can include the ambient light level, the temperature within the vehicle, the temperature outside of the vehicle, the ambient sound level, and other parameters. The parameters can all be indicative of the individual being tired during the day. For example, the sound within the vehicle being loud (e.g., if the radio is being played at a high volume) can indicate that the individual is attempting to stay awake. In another example, the individual could be running the air conditioning at a high level (e.g., the temperature within the car is relatively much colder than the temperature outside of the car) in an attempt to stay awake. The light level within the car being increased could indicate that the individual has turned on one or more lights within the car to try and stay awake.

The third category of parameters includes parameters associated with the vehicle itself. The parameters in this category all generally relate to movement of the vehicle during while being operated, or different functions of the vehicle, and. These parameters can include the volume of audio being played by the vehicle (which is generally a narrower version of the ambient sound level parameter in the second category above), the status of a seat massaging function of the vehicle, a reclining angle of the individual's seat in the vehicle, whether the vehicle is the individual's primary vehicle, the speed of the vehicle, an acceleration of the vehicle, a braking frequency of the vehicle, a braking force of the vehicle, and amount of lateral movement of the vehicle, and others. The parameters related to settings of the vehicle can indicate that the individual is attempting to stay awake. For example, the individual playing the vehicle audio (e.g., radio or music) at a high volume, having a seat massaging function activated, or having the seat at a relatively upright angle can indicate that the individual is trying to stay awake. If these are occurring during the daytime, this can indicate that the individual is experiencing daytime tiredness, which can increase the risk of having or developing SDB. The parameters related to the movement of the vehicle can indicate if the individual is having difficulty staying awake while driving. For example, braking too frequently, braking with too much force, accelerating irregularly, and swerving within our outside of the vehicle's lane can all indicate that the individual is tired and is having difficulty controlling the vehicle. The parameter related to whether the vehicle is the individual's primary vehicle can be used in some implementations as a check on the other parameters. If the vehicle is not the individual's primary vehicle (e.g., a rental car, a spouse's car, etc.), other parameters may not as strongly indicate the individual's risk of developing or having the condition. For example, the individual may operate the brakes too frequently or with too much force if they are not used to driving the vehicle. Thus, while frequent and/or forceful braking may otherwise indicate that the individual is exhibiting signs of daytime tiredness, they do not here, because the vehicle is not the individual's primary vehicle.

The fourth category of parameters includes parameters related to the driving sessions themselves. These parameters can include an origin and a destination of the vehicle for each driving session, a distance traveled during each driving session, a time span of each driving session, the frequency of breaks taken during each driving session, the time span of any breaks taken during each driving session, a beginning time and an ending time of each driving session (which could be used to determine the time span of each driving session), and other parameters. In some cases, a sedentary lifestyle can increase the risk that the individual will develop SDB. Thus, these parameters can be analyzed to determine if the driving sessions indicate that the individual is relatively sedentary. For example, longer driving sessions with relatively few breaks can indicate that the individual is sedentary and at an increased risk of developing SDB.

Generally, each of these parameters has a value. The value of the parameters could be a numerical quantity or range (e.g., the individual's neck circumference, the vehicle's current speed, etc.). The value of the parameters could also be a binary value corresponding to either an on state or an off state. Other possible values can also be used. The values of the parameters can be analyzed at step 604 in a variety of different ways.

In optional step 604A, the value of any one or more of the parameters is determined for each driving session of the plurality of driving sessions. Thus, if there are 10 different parameters being analyzed and 10 different driving sessions, there will be 100 unique parameter values. In some implementations, the absolute value of the parameters is determined. In other implementations, a relative value of the parameters is determined. This relative value could be relative to any suitable baseline, such as a baseline value of the individual, a baseline value for a population to which the individual belongs, etc. The population to which the individual belongs can be based on a number of different factors, such as age, height, weight, sex, medical conditions, occupation, driving frequency, etc. In some implementations, the parameter values are all determined in the same way (e.g., all absolute values, all values relative to the individual's baseline, all values relative to a population baseline, etc.). In other implementations, the values of different parameters can be determined differently (e.g., absolute value for some parameters, values relative to the individual's baseline for other parameters, values relative to a population baseline for further parameters, etc.).

The baseline value for the individual could change depending on the circumstances. For example, in some implementations, the baseline value for the individual is a baseline value for when it was known that the individual had not developed SDB and/or was not at risk for developing SDB. Similarly, the baseline value for the individual's population could include only individuals who are known to not have SDB, and/or to not be at risk of developing SDB. In other implementations, the baseline value for the individual is a simple rolling average value of the parameter across one or more driving sessions, regardless of whether the individual had SDB or was at risk of developing SDB at the time of any of those driving sessions. Similarly, the baseline value for the individual's population could simply be a rolling average value of the parameter across multiple individuals and/or driving sessions, regardless of whether any of the individuals had SDB or were at risk of developing SDB during any of those driving sessions.

In optional step 604B, a change in the value of one or more parameters between two driving sessions is determined. Generally, the change in the value between the two driving sessions is the difference between the values. In some cases, a single change in the value of the parameter between two driving sessions is determined. In other cases, multiple changes in the value of the parameter between two driving sessions can be determined. For example, if there are three different driving sessions being analyzed, step 604B could include determining the change in parameter value between the first driving session and the second driving session, and between the second driving session and the third driving session. In further cases, step 604B includes determining only one change in value for some parameters, and multiple changes in value for other parameters. In some cases, the two driving sessions over which the change in value is determined are consecutive driving sessions. In other cases, the two driving session over which the change in value is determined are not consecutive driving sessions, and are separated by at least one additional driving session.

In optional step 604C, the rate of change in the value of one or more parameters across at least two of the driving sessions is determined. In some implementations, the rate of change is the average change in the value per driving session. In other implementations, the rate of change is the average change in the value per unit of time. The rate of change could also be a rolling average change per driving session or per unit of time. In implementations where the rate of change is the average change in the value per driving session across two driving sessions, the rate of change in step 604C will generally be the same as the change between the two driving sessions in step 604B. whether the individual is operating the vehicle or not. In these implementations, a first weighting can be applied to parameter values (e.g., the raw values, changes in values, rates of changes in values, or other quantifications of the parameters) associated with driving sessions where the individual operated the vehicle, and a second weighting can be applied to parameter values associated with driving sessions. The first and second weightings are generally different from each other, and will generally include distinct values for each different parameter.

Moreover, some parameters are weighted more heavily if the individual is driving, while other parameters are weighted more heavily if the individual is not driving. For example, if the individual is driving, the reclining angle of the driver's seat may not be weighted as heavily, because the individual cannot recline their seat very much, regardless of whether they are tired. However, if the individual is not driving and in a passenger's seat, the reclining angle of the passenger's seat may be more indicative of the individual's risk factor, because the individual is free to recline the passenger's seat at any angle. In another example, the movement of the vehicle can be weighted more heavily when the individual is driving as compared to when the individual is not driving.

Finally, at step 606 of method 600, the individual's risk factor for the condition can be determined based at least in part on the analyzed data. In some implementations the risk factor is indicative of the onset of a condition or the severity of a condition. Thus, the risk factor can indicate to the individual whether they are in danger of developing the condition, or how severe the condition is if the individual has already developed the condition. In some implementations, the risk factor is a percentage likelihood that the individual will develop condition, or has already developed the condition. In some implementations, the risk factor is an estimated time period within which the individual will develop the condition.

In some implementations, the data is analyzed multiple different ways to determine the risk factor of the individual. For example, determining the individual's risk factor at step 606 can be based on the value of one or more parameters (step 604A) and the change in the value of one or more parameters (step 604B), the value of one or more parameters (step 604A) and the rate of change in the value of one or more parameters (step 604C), or all three. Additional or alternative methods of analysis can be used on the data to then determine the individual's risk factor.

In some cases, step 606 includes inputting the parameter values, changes in values, rates of change of values, and/or any other data into a trained model. The trained model can process these inputs and output the risk factor. The model can be trained using a training data set that has been collected from individuals who have developed SDB (or another condition of interest). Generally, the training data set for each individual will include various parameter values, changes in values, and rates of changes of values, and some indication as to the temporal relation between when the inputs were obtained, and when the individual developed the condition. Thus, for a given individual, the training data set can include parameters obtained from a number of driving sessions, where the parameters include some sort of timestamp indicating when the parameters were generated relative to when the individual developed the condition.

Any suitable model can be trained and used to generate the risk factor. In some implementations, the model is an artificial neural network that is trained using any suitable training algorithm(s). Other models can also be used however, such as linear regression models, naive Bayes classifiers, clustering-based models, etc.

As shown in FIG. 6, method 600 can also include optional steps 608 and 610. In optional step 608, data associated with the individual outside of the plurality of driving sessions is generated. In optional step 610, the risk factor is adjusted based on this outside data. The outside data can include any relevant data that is not generally associated with any of the driving sessions, such as demographic information associated with the individual (e.g., age, gender, race, family medical history, etc.), sleep data associated with the individual (e.g., sleep-related parameters associated with one or more sleep sessions of the individual), activity data associated with any activity or exercise done by the individual, food data associated with food eaten by the individual, calendar data associated with entries in the individual's calendar, and a variety of other data.

In some cases, the outside data can indicate that the individual's risk factor is incorrect. For example, if the individual was very active the day prior to a driving session (e.g., the individual went for a hike) and did not sleep much that night, the data from a driving session the next day may indicate that the individual is experiencing daytime tiredness. In this case however, the daytime tiredness is not an indication that the individual is developing SDB (or another condition), but instead only because the individual was very active the day prior and did not get much sleep (for reasons unrelated to the condition). Thus, the risk factor can be adjusted based on this outside data, to account for the individual's activity and lack of sleep.

Generally, method 600 is shown as determining the individual's risk factor based on data from a plurality of driving sessions. However, in some implementations, the determination of the risk factor can be based on data from only a single driving session.

Method 600 can include additional steps. For example, in some implementations, method 600 includes taking an action based at least in part on the individual's risk factor for the condition. In some cases, the action can include notifying the individual of the determined risk factor, and recommending follow-up action by the individual. The follow-up action could include visiting a healthcare provider, beginning treatment with a respiratory therapy system (such as respiratory therapy system 100 of system 10), updating treatment settings of a respiratory therapy system if the individual is already using a respiratory therapy system, and other actions.

Method 600 could also include collecting feedback data in order to perform a check on the determined risk factor. For example, method 600 could include prompting the individual to answer questions related to the driving sessions, or to otherwise provide data related to the driving sessions (for example by inputting information about the driving sessions into a device, such as the user device 260 of system 10). Based on the feedback data, the individual's risk factor can be checked to ensure the risk factor is accurate, and then updated if necessary.

Generally, method 600 can be implemented using a system having a control system with one or more processors, and a memory storing machine readable instructions. The controls system can be coupled to the memory, and method 600 can be implemented when the machine readable instructions are executed by at least one of the processors of the control system. Method 600 can also be implemented using a computer program product (such as a non-transitory computer readable medium) comprising instructions that when executed by a computer, cause the computer to carry out the steps of method 600.

In some implementations, the vehicle is a rideshare vehicle that the individual does not own, and that is also driven by other individuals. In these implementations, much of the data associated with the driving sessions will be collected by the individual's mobile device, so that the individual's driving sessions can still be monitored. In some cases, the individual may have a key fob that the individual takes with them when the drive the vehicle. The key fob may be able to generate, collect, and store data associated with the driving sessions.

### Alternative Implementations

Implementation 1. A method for determining a likelihood that an individual has or will develop a condition, the method comprising: generating, during a plurality of driving sessions, data associated with the plurality of driving sessions, the individual being located within a vehicle during at least a portion of each of the plurality of driving sessions; analyzing the data; and determining, based at least in part on the analyzed data, a risk factor for the individual associated with the condition.

Implementation 2. The method of implementation 1, wherein the data associated with the plurality of driving sessions includes one or more parameters associated with each of the plurality of driving sessions.

Implementation 3. The method of implementation 2, wherein analyzing the data includes determining a value of each of the one or more parameters for each of the plurality of driving sessions, and wherein the risk factor is determined based at least in part on the determined value of each of the one or more parameters for at least one of the plurality of driving sessions.

Implementation 4. The method of implementation 2 or implementation 3, wherein analyzing the data includes determining a change in the value of at least one of the one or more parameters between a first one of the plurality of driving sessions and a second one of the plurality of driving sessions, and wherein the risk factor is determined based at least in part on the determined change in the value of the at least one of the one or more parameters.

Implementation 5. The method of any one of implementations 2 to 4, wherein analyzing the data includes determining a rate of change of the value of at least one of the one or more parameters across two or more of the plurality of driving sessions, and wherein the risk factor is determined factor based at least in part on the determined range of change in the value of the at least one of the one or more parameters.

Implementation 6. The method of any one of implementations 2 to 5, wherein the one or more parameters for each of the plurality of driving sessions includes one or more parameters associated with a body of the individual, one or more parameters associated with an environment around the individual during the plurality of driving sessions, one or more parameters associated with the vehicle, one or more parameters associated with the plurality of driving sessions, or any combination thereof.

Implementation 7. The method of implementation 6, wherein the one or more parameters associated with the body of the individual includes a posture of the individual, a weight of the individual, a neck circumference of the individual, a waist circumference of the individual, a ratio of the neck circumference of the individual to the waist circumference of the individual, an amount of eye movement of the individual, a speed of eye movement of the individual, an amount of blinking by the individual, an amount of head movement of the individual, a speed of head movement of the individual, a direction of head movement of the individual, a weight distribution of the individual in a seat of the vehicle, a strength of a grip of the individual on a steering wheel of the vehicle, or any combination thereof.

Implementation 8. The method of implementation 6 or implementation 7, wherein the one or more parameters associated with the environment around the individual during the plurality of driving sessions includes an ambient light level, a temperature within the vehicle, a temperature outside of the vehicle, an ambient sound level, or any combination thereof.

Implementation 9. The method of any one of implementations 6 to 8, wherein the one or more parameters associated with the vehicle include a volume of audio being played by the vehicle, a status of a seat massager of the vehicle, a reclining angle of a seat of the individual, whether the vehicle is a primary vehicle of the individual, a speed of the vehicle, an acceleration of the vehicle, a braking frequency of the vehicle, an amount of lateral movement of the vehicle, or any combination thereof.

Implementation 10. The method of any one of implementations 6 to 9, wherein the one or more parameters associated with the plurality of driving sessions include an origin of the vehicle for each of the plurality of driving sessions, a destination of the vehicle for each of the plurality of driving sessions, a time span of each of the plurality of driving sessions, a distance traveled during each of the plurality of driving sessions, a frequency of breaks taken during each of the plurality of driving sessions, a length of breaks taken during each of the plurality or driving sessions, a beginning time of each of the plurality of driving sessions, an ending time of each of the plurality of driving sessions, or any combination thereof.

Implementation 11. The method of any one of implementations 1 to 10, wherein during each of the plurality of driving sessions, the individual is operating the vehicle or is a passenger within the vehicle.

Implementation 12. The method of implementation 11, wherein analyzing the data associated with the plurality of driving sessions includes applying a first weighting to data generated during driving sessions where the individual operates the vehicle, and applying a second weighting to data generated during driving sessions where the individual is a passenger within the vehicle, the first weighting being different than the second weighting.

Implementation 13. The method of any one of implementations 1 to 12, wherein each driving session begins when the individual enters the vehicle at an origin, and ends when the individual exits the vehicle at a destination.

Implementation 14. The method of implementation 13, wherein each driving session includes at least a first amount of time when the vehicle is moving and a second amount of time when the vehicle is not moving.

Implementation 15. The method of implementation 13, wherein each driving session includes only time when the vehicle is moving.

Implementation 16. The method of any one of implementations 1 to 15, further comprising: generating data associated with the individual outside of the plurality of driving sessions; and adjusting the risk factor based at least in part on the data associated with the individual outside of the driving sessions.

Implementation 17. The method of implementation 16, wherein the data associated with the individual outside of the plurality of driving sessions includes activity data associated with the individual, sleep data associated with the individual, calendar data associated with the individual, food data associated with the individual, or any combination thereof.

Implementation 18. The method of any one of implementations 1 to 17, wherein the risk factor is indicative of an onset of the condition or of a severity of the condition.

Implementation 19. The method of any one of implementations 1 to 18, wherein the risk factor includes a percentage likelihood that the individual will develop the condition, an estimated time period within which the individual will develop the condition, a percentage likelihood that the individual currently has the condition, or any combination thereof.

Implementation 20. The method of any one of implementations 1 to 19, wherein the condition includes sleep-disordered breathing (SDB), allergies, a cold, a dry throat, a sore throat, a viral infection, pneumonia, chronic obstructive pulmonary disease (COPD), asthma, stroke, enlarged tonsils, high blood pressure, low blood pressure, or any combination thereof.

Implementation 21. The method of any one of implementations 1 to 20, wherein the data is generated by one or more sensors located within the vehicle, the one or more sensors including at least one image sensor.

Implementation 22. The method of implementation 21, wherein the at least one image sensor includes a camera coupled to a rear-view mirror of the vehicle and facing toward the individual when the individual is located within the vehicle.

Implementation 23. The method of implementation 21 or implementation 22, wherein the at least one image sensor includes a camera facing away from the individual toward an exterior of the vehicle.

Implementation 24. The method of any one of implementations 1 to 23, wherein the data is generated by a mobile device of the individual.

Implementation 25. The method of any one of implementations 1 to 23, wherein the data is generated by a control system of the vehicle.

Implementation 26. A system for determining a likelihood that an individual has or will develop a condition, the system comprising: a control system including one or more processors; and a memory having stored thereon machine-readable instructions; wherein the control system is coupled to the memory, and the method of any one of implementations 1 to 25 is implemented when the machine-readable instructions in the memory are executed by at least one of the one or more processors of the control system.

Implementation 27. A system for determining a sleep stage of an individual, the system including a control, the system including a control system having one or more processors configured to implement the method of any one of implementations 1 to 25.

Implementation 28. A computer program product comprising instructions which, when executed by a computer, cause the computer to carry out the method of any one of implementations 1 to 25.

Implementation 29. The computer program product of implementation 28, wherein the computer program product is a non-transitory computer readable medium.

One or more elements or aspects or steps, or any portion(s) thereof, from one or more of any of claims can be combined with one or more elements or aspects or steps, or any portion(s) thereof, from one or more of any of the other claims or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method for determining a likelihood that an individual has or will develop a condition, the method comprising:
generating, during a plurality of driving sessions, data associated with the plurality of driving sessions, the individual being located within a vehicle during at least a portion of each of the plurality of driving sessions;
analyzing the data; and
determining, based at least in part on the analyzed data, a risk factor for the individual associated with the condition.

2. The method of claim 1, wherein the data associated with the plurality of driving sessions includes one or more parameters associated with each of the plurality of driving sessions, and wherein analyzing the data includes determining a value of each of the one or more parameters for each of the plurality of driving sessions, and wherein the risk factor is determined based at least in part on the determined value of each of the one or more parameters for at least one of the plurality of driving sessions.

3. (canceled)

4. The method of claim 2, wherein analyzing the data includes:
(i) determining a change in the value of at least one of the one or more parameters between a first one of the plurality of driving sessions and a second one of the plurality of driving sessions, and wherein the risk factor is determined based at least in part on the determined change in the value of the at least one of the one or more parameters;
(ii) determining a rate of change of the value of at least one of the one or more parameters across two or more of the plurality of driving sessions, and wherein the risk factor is determined factor based at least in part on the determined range of change in the value of the at least one of the one or more parameters; or (iii) both (i) and (ii).

5. (canceled)

6. The method of claim 2, any wherein the one or more parameters for each of the plurality of driving sessions includes one or more parameters associated with a body of the individual, one or more parameters associated with an environment around the individual during the plurality of driving sessions, one or more parameters associated with the vehicle, one or more parameters associated with the plurality of driving sessions, or any combination thereof.

7. The method of claim 6, wherein the one or more parameters associated with the body of the individual includes a posture of the individual, a weight of the individual, a neck circumference of the individual, a waist circumference of the individual, a ratio of the neck circumference of the individual to the waist circumference of the individual, an amount of eye movement of the individual, a speed of eye movement of the individual, an amount of blinking by the individual, an amount of head movement of the individual, a speed of head movement of the individual, a direction of head movement of the individual, a weight distribution of the individual in a seat of the vehicle, a strength of a grip of the individual on a steering wheel of the vehicle, or any combination thereof.

8. The method of claim 6, wherein the one or more parameters associated with the environment around the individual during the plurality of driving sessions includes an ambient light level, a temperature within the vehicle, a temperature outside of the vehicle, an ambient sound level, or any combination thereof.

9. The method of claim 6, wherein the one or more parameters associated with the vehicle include a volume of audio being played by the vehicle, a status of a seat massager of the vehicle, a reclining angle of a seat of the individual, whether the vehicle is a primary vehicle of the individual, a speed of the vehicle, an acceleration of the vehicle, a braking frequency of the vehicle, an amount of lateral movement of the vehicle, or any combination thereof.

10. The method of claim 6, wherein the one or more parameters associated with the plurality of driving sessions include an origin of the vehicle for each of the plurality of driving sessions, a destination of the vehicle for each of the plurality of driving sessions, a time span of each of the plurality of driving sessions, a distance traveled during each of the plurality of driving sessions, a frequency of breaks taken during each of the plurality of driving sessions, a length of breaks taken during each of the plurality or driving sessions, a beginning time of each of the plurality of driving sessions, an ending time of each of the plurality of driving sessions, or any combination thereof.

11. The method of claim 1, wherein during each of the plurality of driving sessions, the individual is operating the vehicle or is a passenger within the vehicle, and wherein analyzing the data associated with the plurality of driving sessions includes applying a first weighting to data generated during driving sessions where the individual operates the vehicle, and applying a second weighting to data generated during driving sessions where the individual is a passenger within the vehicle, the first weighting being different than the second weighting.

12. (canceled)

13. The method of claim 1, any wherein each driving session begins when the individual enters the vehicle at an origin, and ends when the individual exits the vehicle at a destination.

14. The method of claim 13, wherein each driving session includes at least a first amount of time when the vehicle is moving and a second amount of time when the vehicle is not moving, or wherein each driving session includes only time when the vehicle is moving.

15. (canceled)

16. The method of claim 1, further comprising:
generating data associated with the individual outside of the plurality of driving sessions; and
adjusting the risk factor based at least in part on the data associated with the individual outside of the driving sessions.

17. The method of claim 16, wherein the data associated with the individual outside of the plurality of driving sessions includes activity data associated with the individual, sleep data associated with the individual, calendar data associated with the individual, food data associated with the individual, or any combination thereof.

18. The method of claim 1, wherein the risk factor is indicative of an onset of the condition or of a severity of the condition, and includes a percentage likelihood that the individual will develop the condition, an estimated time period within which the individual will develop the condition, a percentage likelihood that the individual currently has the condition, or any combination thereof.

19. (canceled)

20. The method of claim 1, any wherein the condition includes sleep-disordered breathing (SDB), allergies, a cold, a dry throat, a sore throat, a viral infection, pneumonia, chronic obstructive pulmonary disease (COPD), asthma, stroke, enlarged tonsils, high blood pressure, low blood pressure, or any combination thereof.

21. The method of claim 1, any wherein the data is generated by one or more sensors located within the vehicle, the one or more sensors including at least one image sensor.

22. The method of claim 1, wherein the at least one image sensor includes a camera coupled to a rear-view mirror of the vehicle and facing toward the individual when the individual is located within the vehicle, a camera facing away from the individual toward an exterior of the vehicle, or both.

23. (canceled)

24. The method of claim 1, any wherein the data is generated by a mobile device of the individual, by a control system of the vehicle, or both.

25. (canceled)

26. A system for determining a likelihood that an individual has or will develop a condition, the system comprising:
a control system including one or more processors; and
a memory having stored thereon machine-readable instructions;
wherein the control system is coupled to the memory, and the method of claim 1 is implemented when the machine-readable instructions in the memory are executed by at least one of the one or more processors of the control system.

27. A system for determining a likelihood that an individual has or will develop a condition, the system including a control system having one or more processors configured to implement the method of claim 1.

28-29. (canceled)
